(19) United States
(12) Patent Application Publication
(10) Pub. No.: US 2025/0177429 A1
(43) Pub. Date: Jun. 5, 2025

(54) ORALLY ACTIVE PRODRUG OF GEMCITABINE

(71) Applicant: Laevoroc Chemotherapy AG, Huenenberg (CH)

(72) Inventors: Davide Guggi, Vienna (AT); Didier Foechterlen, Rimbach-Zell (FR)

(21) Appl. No.: 18/842,883

(22) PCT Filed: Mar. 10, 2023
(86) PCT No.: PCT/EP2023/056196

§ 371 Date: Aug. 30, 2024

(30) Foreign Application Priority Data
Mar. 11, 2022 (GB) 2203421.9

(51) Int. Cl. A61K 31/7068; C07H 1/06; C07H 19/073

(52) U.S. Cl. CPC A61K 31/7068; C07H 1/06; C07H 19/073

(57) ABSTRACT

The disclosure includes compounds in accordance with Formula (I) or Formula (II) wherein R₁, R₂, and R₃ are defined herein and to methods of making those compounds. That disclosure extends 5 to those compounds for the treatment of neoplastic disease, or a method for treating neoplastic disease with these compounds.

### BACKGROUND

Gemcitabine, depicted below, is a pyrimidine nucleoside analogue, shown to be active against several solid tumortypes. Following FDA approval in 1996, gemcitabine has become the standard of care for the treatment of pancreatic cancer. More recently, the compound has also gained approval for treating non-small cell lung, ovarian, bladder, and breast cancer.

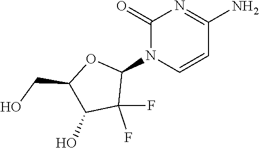 [SMILES: Nc1ccn([C@@H]2O[C@H](CO)[C@@H](O)C2(F)F)c(=O)n1]

### The Chemical Structure of Gemcitabine

Gemcitabine is administered by intravenous infusion at a dose of approximately 1000 to 1250 mg/m² over 30 minutes, once weekly for up to 7 weeks followed by a week of rest from treatment. The use of gemcitabine by oral means is limited by its poor oral bioavailability, which is the result of first pass metabolism. Shipley L A. Et. al., “Metabolism and disposition of gemcitabine, and oncolytic deoxycytidine analog, in mice, rats, and dogs”. Drug Metabolism & Disposition. 20(6):849-55, 1992. In addition, when dosed orally, gemcitabine is implicated in causing adverse dose-limiting intestinal lesions characterized by moderate-to-marked loss of mucosal epithelium (atrophic enteropathy) throughout the entire length of the intestinal tract in mice given a single oral (gavage) gemcitabine dose of 167, 333, or 500 mg/kg. Horton N D et. al., “Toxicity of single-dose oral gemcitabine in mice”, American Association for Cancer Research, Poster Presentation, Orlando, FL, Mar. 27-31, 2004. Comparable exposures via intravenous dosing in previous mouse studies did not result in death or gastrointestinal toxicity.

In 2009, Bender et al. reported an orally active prodrug of gemcitabine; LY2334737, was significantly less prone to degradation by CDA due to a valproic acid linkage at the 4-(N)-position. Based on in vivo data in the HCT-116 human colon xenograft, LY2334737 was advanced into phase I clinical studies but the development was terminated after unexpected hepatic toxicities were observed in a study of Japanese patients in 2013.

As shown in the gemcitabine chemical structure above, gemcitabine has three functional groups (i.e. —OH, —OH, —NH₂) that are amenable to chemical prodrug derivatization. A triple drug is described in WO2020/107013, in which all three available moieties are substituted. However, the triple substituted pro-drug compound is unstable at room temperature. The instability of this active ingredient presented a significant problem in formulation development and a drug product did not result to be stable. The triple substituted compound is therefore unsuitable for progressing as an orally active prodrug for therapeutic use.

There is a continuing search in this field of art for orally active prodrug of gemcitabine for development and/or to provide a medicinal product treatment suitable for cancer therapy and in particular, for use in the treatment of pancreatic, ovarian and breast cancers.

### SUMMARY OF THE INVENTION

The present invention relates to a novel class of orally active prodrugs of Gemcitabine comprising an orally active substituted-prodrug, in which a combination of specific functional groups are derivatized with a pro-moiety (a chemical functional group used to modify the structure of parent drug to improve physicochemical, biopharmaceutical or pharmacokinetic properties).

The pro-moiety is typically inactive and biologically safe. However, one of these groups is typically always H, specifically the group at R₃ position, as shown below.

This invention therefore provides compounds of the Formula (I), or an N-oxide thereof, or a pharmaceutically acceptable salt, solvate, polymorph, tautomer, stereoisomer, an isotopic form, or a prodrug of said compound of Formula (I) or N-oxide thereof:

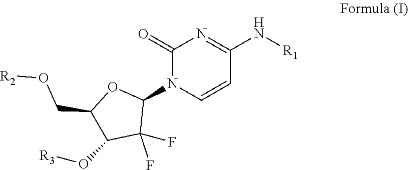

wherein
R₁ is selected from 2-propylpentanoic acid, 2-(tert-butoxycarbonyl)-amino-3-methylbutanoic acid, and 2-amino-3-methylbutanoic acid;
R₂ is H; and
R₃ is selected from H, 2-propylpentanoic acid, 2-(tert-butoxycarbonyl)-amino-3-methylbutanoic acid, and 2-methylpropanoic acid.

This substituted compound has one free primary alcohol position (no isobutyric moiety) and it advantageous because it shows increased stability for the formulation of a medicament suitable for therapeutic use.

In other embodiments, R₁ and R₃ are selected in the following respective combinations to yield the specifically named compounds:
Valproic acid (2-propylpentanoic acid) (R₁) and Valproic acid (R₃), the compound of that embodiment being: (2R,3R,5R)-4,4-difluoro-2 (hydroxymethyl)-5-(2-oxo
4-(2propylpentanamido)-1,2-dihydropyrimidin-1-yl)oxolan-3-yl 2-propylpentanoate; or
Valproic acid and H, the compound of that embodiment being N-(1-((2R,4R,5R)-3,3-difluoro-4-hydroxy-5-(hydroxymethyl)oxolan-2-yl)-2-oxo-1,2-dihydropyrimidin-4-yl)-2-propylpentanamide; or
Boc Valine (2-(tert-butoxycarbonyl)-amino-3-methylbutanoic acid) and H, the compound of that embodiment being: tert-butyl (1-((1-((2R,4R,5R)-3,3-difluoro-4-hydroxy-5-(hydroxymethyl)oxolan-2-yl)-2-oxo-1,2-dihydropyrimidin-4-yl)amino)-3-methyl-1-oxobutan-2-yl)carbamate; or
Valine (2-amino-3-methylbutanoic acid) and H, the compound of that embodiment being 2-amino-N-(1-((2R,4R,5R)-3,3-difluoro-4-hydroxy-5-(hydroxymethyl)oxolan-2-yl)-2-oxo-1,2-dihydropyrimidin-4-yl)-3-methylbutanamide hydrochloride; or
BocValine and BocValine, the compound of that embodiment being (2R,3R,5R)-5-(4-(2-((tert-butoxycarbonyl)amino)-3-methylbutanamido)-2-oxo-1,2-dihydropyrimidin-1-yl)-4,4-difluoro-2-(hydroxymethyl)oxolan-3-yl (2S)-2-(tert-butoxycarbonyl) amino-3-methylbutanoate;
Valine and Valine, the compound of that embodiment being (2R,3R,5R)-5-(4-(2-amino-3-methylbutanamido)-2-oxo-1,2-dihydropyrimidin-1-yl)-4,4-difluoro-2-(hydroxymethyl)oxolan-3-yl (2S)-2-amino-3-methylbutanoate dihydrochloride; or
BocValine and Isobutyric acid (2-methylpropanoic acid), the compound of that embodiment being (2R,3R,5R)-5-(4-(2-((tert-butoxycarbonyl)amino)-3-methylbutanamido)-2-oxo-1,2-dihydropyrimidin-1-yl)-4,4-difluoro-2-(hydroxymethyl)oxolan-3-yl isobutyrate; or
Valine and Isobutyric acid, the compound of that embodiment being (2R,3R,5R)-5-(4-(2-amino-3-methylbutanamido)-2-oxo-1,2-dihydropyrimidin-1-yl)-4,4-difluoro-2-(hydroxymethyl)oxolan-3-yl isobutyrate hydrochloride; or
Valproic acid and Isobutyric acid, the compound of that embodiment being (2R,3R,5R)-4,4-difluoro-2-(hydroxymethyl)-5-(2-oxo-4-(2-propylpentanamido)-1,2-dihydropyrimidin-1-yl)oxolan-3-yl isobutyrate.

In embodiments, the pro-drug moiety at R₃ is not H.

In further embodiments, the compound is represented by Formula (II):

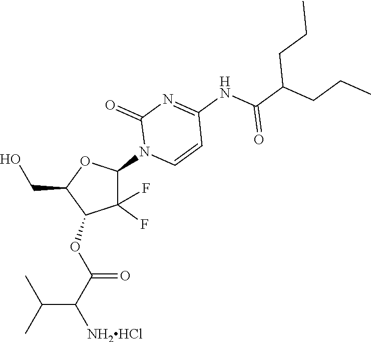

and/or the compound named (2R,3R,5R)-4,4-difluoro-2-(hydroxymethyl)-5-(2-oxo-4-(2-propylpentanamido)-1,2-dihydropyrimidin-1-yl)oxolan-3-yl (2S)-2-amino-3-methylbutanoate hydrochloride.

The double substituted compound has improved stability at room temperature. Further, the compound of the invention has stability from 2° C. to 8° C. As compared to the triple substituted compound, the invention has essential characteristics and advantages. The compound of the invention is sufficiently stable to be useful as an active ingredient, particularly as an API in formulations, enabling both stable storage and transportation, which are key factors in the provision of a commercially suitable medicinal product.

The invention extends to a pharmaceutical composition, comprising a formulation including the compound disclosed herein.

In embodiments, the formulation comprises a powder. The invention further extends to a capsule comprising the formulation.

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof. In embodiments, the preferred salt is a hydrochloride salt.

Also, within the scope of this invention is the compound and/or a pharmaceutical composition containing one or more of the compounds and/or salts thereof described, for use in the treatment of a neoplastic disease including a pim-overexpressed neoplastic disease including but not limited to, leukemia, lymphoma, multiple myeloma, prostate cancer, pancreatic cancer, gastric cancer, colon cancer, or liver cancer.

The invention further extends to use of the compounds in the manufacture of a medicament for treating such disease. This invention also relates to a method of treating a Pim-overexpressed neoplastic disease, including but not limited to leukemia, lymphoma, multiple myeloma, prostate cancer, pancreatic cancer, gastric cancer, colon cancer, or liver cancer by administering to a subject in need thereof an effective amount of one or more of the compounds, modifications, and/or salts, and compositions thereof described above.

The invention further extends to a method of producing the compound of the invention, as provided in Formula (I) or (II) or any example disclosed herein, comprising a 4-step process comprising:
selective protection of the primary alcohol of Gemcitabine at R₂ position to produce a first intermediate (1);
peptide coupling of Intermediate 1 at R₁ position to produce a second intermediate (2); esterification of the second intermediate (2) at R₃ position to produce a third intermediate (3); and
deprotection of the third intermediate (3), preferably at room temperature.

In embodiments, the protection of primary alcohol (R2-group) may be undertaken with silylated reactants (TBDMSCl). Further, the step of peptide coupling on the amino group (R1-group) may be performed using EDCl.HCl/HOBt, preferably, in absence of additional base. The additional base that is absent may be DIPEA or TEA, for example. The peptide-like esterification on the secondary alcohol (R3-group) may be undertaken using EDCl.HCl/HOBt as coupling agent, optionally using DMAP as catalyst. Finally, the deprotecting step maybe undertaken with either 4M HCl/dioxane or AcCl/EtOH.

In embodiments, the method further comprises an isolation step and may be undertaken by performing precipitation from the reaction mixture using an anti-solvent. Preferably, TBME is used as anti-solvent.

This resulting method/pathway of the invention creates a new compound unexpectedly enables such production with fewer synthetic steps. In particular, at least the chromatographic step previously assumed and associated to be essential, i.e. those required to produce the tri-substituted compounds and related compounds of the prior art, is avoided and this absolves the use of large quantities of solvents and waste (environmental improvement). Further still, expected better final API and formulation stability and identical biological deprotection of the pro-drug in vivo are achieved.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:

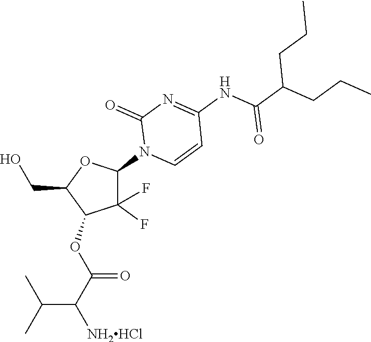

(2R,3R,5R)-4,4-difluoro-2-(hydroxymethyl)-5-(2-oxo-4-(2-propylpentanamido)-1,2-dihydropyrimidin-1-yl)oxolan-3-yl (2S)-2-amino-3-methylbutanoate hydrochloride.

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. Prodrug derivatives: prodrugs, upon administration to a subject, will converted in vivo into active compounds of the present invention [Nature Reviews of Drug Discovery, 2008, Volume 7, p255]. It is noted that in many instances, the prodrugs themselves also fall within the scope of the range of compounds according to the present invention. The prodrugs of the compounds of the present invention can be prepared by standard organic reaction, for example, by reacting with a carbamylating agent (e.g., 1,1-acyloxyalkylcarbonochloridate, para-nitrophenyl carbonate, or the like) or an acylating agent. Further examples of methods and strategies of making prodrugs are described in Bioorganic and Medicinal Chemistry Letters, 1994, Vol. 4, p. 1985. Deuterium-enriched compounds: deuterium (D or 2H) is a stable, non-radioactive isotope of hydrogen and has an atomic weight of 2.0144. Hydrogen naturally occurs as a mixture of the isotopes XH (hydrogen or protium), D (2H or deuterium), and T (3H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In embodiments the pharmaceutically acceptable salt is a hydrochloride salt. In other embodiments the salt is selected from hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form. In embodiments, the particles are powders.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

### Chemical and Biological Terminology

As used herein, “acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

“alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

“alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

“alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

“alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl. The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

“heterocycloalkyl” refers to a nonaromatic 5-8 membered monocyclic, 8-12 membered bicyclic, or 11-14 membered tricyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

“heterocycloalkenyl” refers to a nonaromatic 5-8 membered monocyclic, 8-12 membered bicyclic, or 11-14 membered tricyclic ring system having one or more heteroatoms (such as O, N, S, P, or Se) and one or more double bonds.

“aryl” refers to a 6-carbon monocyclic, 10-carbon bicyclic, 14-carbon tricyclic aromatic ring system. Examples of aryl groups include, but are not limited to, phenyl, naphthyl, and anthracenyl. The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, aryl, and heteroaryl mentioned above include both substituted and unsubstituted moieties.

Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valences (unsubstituted). If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. In one embodiment, the salt selected is a hydrochlorise salt.

“Pharmaceutically acceptable carrier” means a non-toxic solvent, dispersant, excipient, adjuvant, or other material that is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-β-cyclodextrin or sulfobutyl ether β-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well-known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention may be used in combination with other pharmaceutically active compounds, or non-drug therapies, preferably compounds that are able to enhance the effect of the compounds of the invention. The compounds of the invention may be administered simultaneously (as a single preparation or separate preparation) or sequentially to the other therapies. In general, a combination therapy envisions administration of two or more drugs/treatments during a single cycle or course of therapy.

In embodiments, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors (Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

The compounds of the invention may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDR/VEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6 Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3 Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TA01, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F3171), ABL1(G250E), ABL1(H396P), ABL1(M351T), ABL1(Q252H), ABL1(T3151), ABL1(Y253F), ALK (C1156Y), ALK(L1196M), ALK (F1174L), ALK (R1275Q), BRAF(V599E), BTK(E41K), CHK2(I157T), c-Kit(A829P), c-KIT(D816H), c-KIT(D816V), c-Kit(D820E), c-Kit(N822K), C-Kit (T6701), c-Kit(V559D), c-Kit(V559D/V654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T6741), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

The compounds disclosed herein may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors (HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

Compounds of the invention may be administered in combination with one or more of other anti-cancer agents that include, but are not limited to, gene therapy, RNAi cancer therapy, chemoprotective agents (e.g., amfostine, mesna, and dexrazoxane), antibody conjugate(e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

The compounds of the invention may be administered in combination with radiation therapy or surgeries. Radiation is commonly delivered internally (implantation of radioactive material near cancer site) or externally from a machine, that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, anti-metabolites, topoisomerase inhibitors, anti-microtubule agents, kinase inhibitors, epigenetic agents, HSP90 inhibitors, PARP inhibitors, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

The invention further provides methods for the prevention or treatment of a neoplastic disease or autoimmune disease. In one embodiment, the invention relates to a method of treating a neoplastic disease or autoimmune disease, in a subject in need of treatment comprising administering to said subject a therapeutically effective amount of a compound of the invention. In one embodiment, the invention further provides for the use of a compound of the invention in the manufacture of a medicament for halting or decreasing a neoplastic disease or autoimmune disease.

In certain embodiments, the neoplastic disease is a lung cancer, head and neck cancer, central nervous system cancer, prostate cancer, testicular cancer, colorectal cancer, pancreatic cancer, liver cancer, stomach cancer, biliary tract cancer, esophageal cancer, gastrointestinal stromal tumor, breast cancer, cervical cancer, ovarian cancer, uterine cancer, leukemia, lymphomas, multiple myeloma, melanoma, basal cell carcinoma, squamous cell carcinoma, bladder cancer, renal cancer, sarcoma, mesothelioma, thymoma, myelodysplastic syndrome, or myeloproliferative disease.

The autoimmune diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barre syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

### Detailed Description of the Invention

To synthesize a compound according to the present invention or embodiments thereof as per Formula (I) or (II) from the starting material (gemcitabine) the general scheme, as shown below, is followed. Necessary starting materials may be obtained by standard procedures of organic chemistry. This process was invented by the applicant to create an improved pathway, which is more efficient to the existing process for producing derivative compounds. It should be understood that the invention is not limited to the particular embodiments shown and described herein. The compounds and processes of the present invention will be better understood in connection with the following representative examples and not limiting of the scope of the invention.

### Chemical Example

Synthesis of an example double substituted gemcitabine product or LR-06-B.HCL chemically known as: (2R,3R,5R)-4,4-difluoro-2-(hydroxymethyl)-5-(2-oxo-4-(2-propylpentanamido)-1,2-dihydropyrimidin-1-yl)oxolan-3-yl (2S)-2-amino-3-methylbutanoate hydrochloride

Due to the variability in the polarity of products/intermediates/side-products during the processing, it is noted that three LCMS methods are used:
ESI+_std_75V with detection by 254 nm
ESI+_unpolar_75V with detection by 254 nm
ESI+_unpolar_75V_1200 Da with detection by 254 nm

Example of 4-step process of manufacture:
1. The first step is the selective protection of the primary alcohol of Gemcitabine at R₂ position with a silyl derivative: TBDMSCL or (−)-Deoxyephedrine, N-(tert-butyldimethylsilyl)-

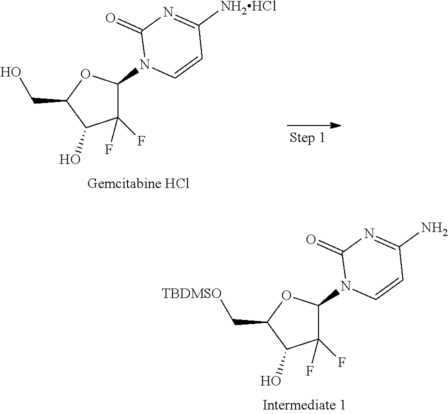

Standard conditions

| | | | |
|---|---|---|---|
| Reactants | Gemcitabine HCl | 1 | eq. |
| | Imidazole | 2 | eq. |
| | TBDMSCI | 1.05 | eq. |

| | | |
|---|---|---|
| Solvent | DMF | 4 mL / g SM |
| Conditions | Temperature | RT (room temperature) |

| | | |
|---|---|---|
| Reaction Time | 2-3 | h (hours) |

| | | Obtained | | | |
|---|---|---|---|---|---|
| Batch Nr. | Scale | Amount | Yield | Appearance | Purity |
| 1935 B1.1 | 30 g | 36.8 g | 97.5% | White solid | 97.9% |
| 1935 B2.1 | 90 g | 114 g | 103% | White solid | 98.0% |
| 1935 B3.1 | 150 g | 196 g | 104% | White solid | 97.0% |

2. The second step is the peptide coupling of Intermediate 1 with valproic acid (peptide coupling) at R₁ position.

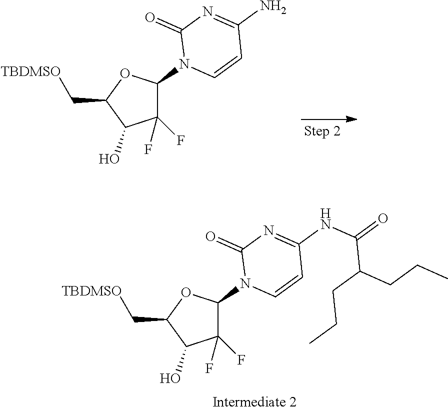 [SMILES: CC(C)(C)[Si](C)(C)OC[C@H]1O[C@@H](n2ccc(N)nc2=O)C(F)(F)[C@@H]1O ; CCCC(CCC)C(=O)Nc1ccn([C@@H]2O[C@H](CO[Si](C)(C)C(C)(C)C)[C@@H](O)C2(F)F)c(=O)n1]

### Standard Conditions

| Reactants | Intermediate 1 | 1 | eq. |
|---|---|---|---|
| | Valproic acid | 1.1 | eq. |
| | EDC•HCl | 1.1 | eq. |
| | HOBt•H₂O | 1.1 | eq. |
| Solvent | ACN | 5 mL / g Intermediate 1 | |
| Conditions | Temperature | 55° | C. |
| | Reaction Time | 6-7 | h |

| | | Obtained | | | |
|---|---|---|---|---|---|
| Batch Nr. | Scale | Amount | Yield | Appearance | Purity |
| 1936 B1.1 | 36.5 g | 47.6 g | 97.5% | White foam | 90% |
| 1936 B2.1 | 114 g | 145.9 g | 95.9% | Pale yellow | 86% |
| | | | | foam | |
| 1936 B3.1 | 98 g | 121.1 g | 92.5% | White foam | 85% |
| 1936 B4.1 | 98 g | 120.2 g | 91.8% | White foam | 88 |

3. The third step is the esterification of Intermediate 2 with Boc-Valine at R₃ position.

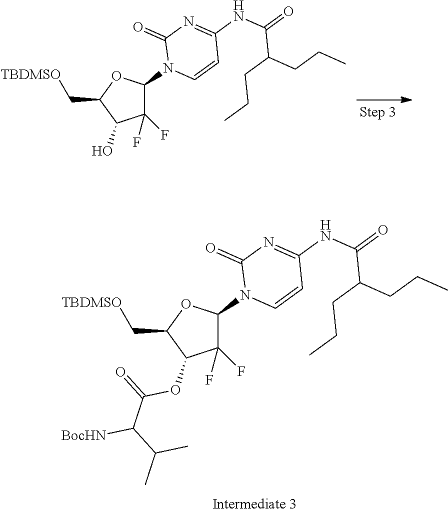 [SMILES: CCCC(CCC)C(=O)Nc1ccn([C@@H]2O[C@H](CO[Si](C)(C)C(C)(C)C)[C@@H](O)C2(F)F)c(=O)n1 ; CCCC(CCC)C(=O)Nc1ccn([C@@H]2O[C@H](CO[Si](C)(C)C(C)(C)C)[C@@H](OC(=O)C(NC(=O)OC(C)(C)C)C(C)C)C2(F)F)c(=O)n1]

Standard conditions

| | | | |
|---|---|---|---|
| Reactants | Intermediate 2 | 1 | eq. |
| | Boc-Valine | 1.1 | eq. |
| | EDC•HCl | 1.1 | eq. |
| | DMAP | 0.1 | eq. |

| | | |
|---|---|---|
| Solvent | ACN | 5 mL / g Intermediate 2 |
| Conditions | Temperature | RT |

| | | |
|---|---|---|
| Reaction Time | 4 | h |

| | | Obtained | | | |
|---|---|---|---|---|---|
| Batch Nr. | Scale | Amount | Yield | Appearance | Purity |
| 1937 B1.1 | 47.6 g | 64.3 g | 97.6% | White foam | 80% |
| 1937 B2.1 | 145.9 g | 201 g | 99% | White foam | 79% |
| 1937 B3.1 | 121.1 g | 170 g | 100% | White foam | 79% |
| 1937 B4.1 | 120.2 g | 170 g | 100% | White foam | 80% |

4. The fourth step comprises deprotection of Intermediate 3 to form a compound (known as LR-06-B.HCL) and in accordance an embodiment of the invention—Formula II.

| | Starting | | | |
|---|---|---|---|---|
| | Material | | | |
| Trial | Purity | Procedure | Yield | Purity |
| 2009-G | Intermediate 3 | Deprotection with 25 eq 4M HCl in dioxane | 81% | 92.7% |
| | 92% (Raw) | Dilution with 2V of Heptane | | |
| 2009-H1 | Intermediate 3 | Deprotection with 25 eq 4M HCl in dioxane | 98% | 97.6% |
| | 98.5% | Dilution with 2V of TBME | | |
| | (Column) | | | |
| 2009-H2 | Intermediate 3 | Deprotection with 25 eq AcCl in Ethanol | 75% | 99.0% |
| | 98.5% | Dilution with 2V of TBME | | |
| | (Column) | | | |
| 2009-I | Intermediate 4 | Deprotection with 25 eq 4M HCl in dioxane | 98% | 92.7% |
| | 87% (Raw) | Dilution with 2V of TBME | | |
| 2009-J1 | Intermediate 4 | Deprotection with 25 eq 4M HCl in dioxane | 100% | 92.7% |
| | (Mix) | Dilution with 2V of TBME | | |
| | 92.5% | | | |
| 2009-J2 | Intermediate 4 | Deprotection with 25 eq 4M HCl in dioxane | 100% | 99.0% |
| | 99.3% | Dilution with 2V of TBME | | |

The resulting twice-substituted gemcitabine example compound, as defined by Formula II and known as LR-06-B.HCL, was easily isolated as a white powder in good yields and comprised the expected purities.

### Spectral Analysis

HPLC-MS was undertaken with the following parameters:
Column: Acquity UPLC® Peptide BEH C18-130 Δ, 1.7 μm-2.1 mm×100 mm
Solvent A: 0.1% HCOOH in Water
Solvent B: 0.1% HCOOH in ACN

| Time (min) | Flow (mL/min) | % A | % B | Curve |
|---|---|---|---|---|
| −5.0 | 0.400 | 95 | 5 | 5 |
| 1.0 | 0.400 | 95 | 5 | 5 |
| 5.0 | 0.400 | 20 | 80 | 5 |
| 8.0 | 0.400 | 20 | 80 | 5 |

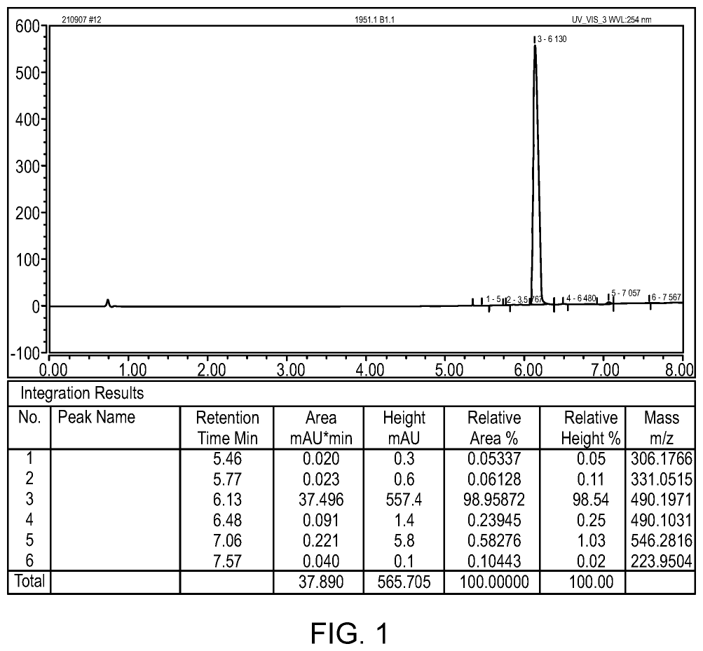

Detection by 254 nm: Rt=6.1 min-m/z=489.2 [M+H]+
Graph for this result is found in FIG. 1

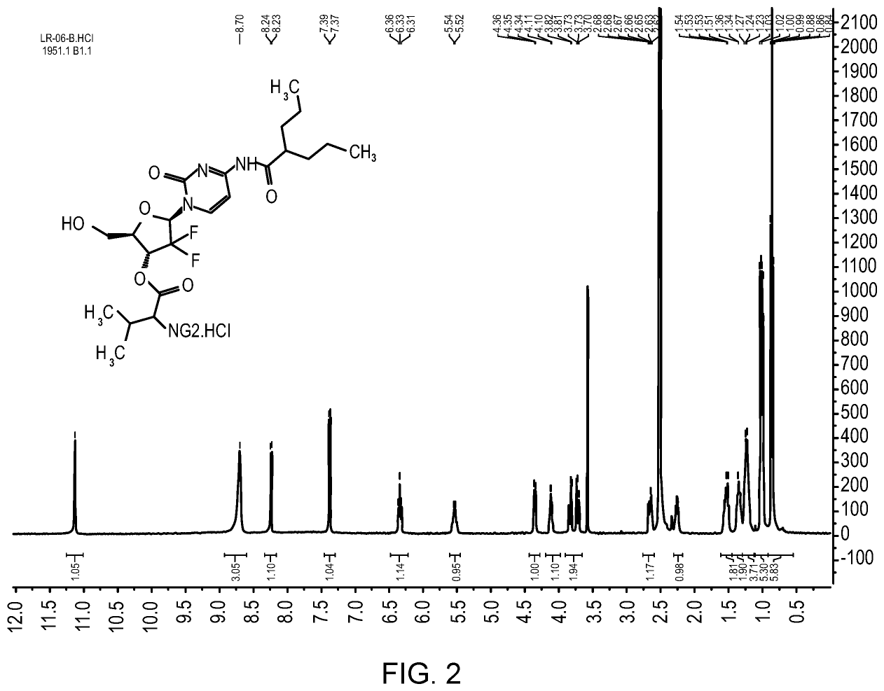

NMR graphic is provided in FIG. 2 with the following raw peak data:

NMR 1H (DMSO-d6, 400 MHz): 11.12 (s, 1H, NH), 8.70 (br s, 3H, NH3+), 8.23 (d, J=7.7 Hz, 1H, CHar), 7.38 (d, J=7.7 Hz, 1H, CHar), 6.33 (t, J=8.4 Hz, 1H, CH), 5.53 (m, 1H, CH), 4.35 (dd, J=7.4 and 3.3 Hz, CH), 4.10 (d, J=4.9 Hz, CH), 3.78 (ddd, J=35, 12.7 and 2.9 Hz, CH2), 2.66 (m, 1H, CH), 2.25 (m, 1H, CH), 1.53 (m, 2H, CH2), 1.35 (m, 2H, CH2), 1.24 (m, 4H, 2×CH2), 1.01 (m, 6H, 2×CH3), 0.86 (m, 6H, 2×CH3).

The melting point was determined to be 203-205° C.

Performed on MP50 Melting point apparatus (Mettler Toledo)

| Measurement | Start T ° | End T ° | Ramp | Result |
|---|---|---|---|---|
| 1 | 20 | 240 | 20° C./min | 207° C. |
| 2 | 200 | 240 | 5° C./min | 205° C. |
| 3 | 200 | 210 | 1° C./min | 203° C. |

### Re-crystallisation

Isolation (by simple filtration, dilution, addition of anti-solvent, cooling) of the preferred example compound made by this procedure was further investigated.

A first set (2010-P, 350 mg scale, purity 97.5%) of recrystalisation tests was performed and appeared promising as the product was always recovered as a good quality powder. In each case, an initial suspension was heated to reflux targeting a full dissolution of the solid. After 10 min heating, the solution or residual suspension was cooled down to RT for 1 hour under stirring before filtration.

| | | Full Dissolution | | |
|---|---|---|---|---|
| Nr. | Solvent & Concentration | (Y/N) | Yield | Purity |
| 1 | Acetone 10 mL/g | No | 92% | 98.7% |
| 2 | Ethanol 10 mL/g | Yes | 40% | 99.5% |
| 3 | TBME 10 mL/g | No | 88% | 97.8% |
| 4 | Acetone 20 mL/g | No | 76% | 98.7% |
| 5 | Ethanol 5 mL/g | No | 48% | 99.6% |

The best combination yield/purity was achieved with acetone. Further, the high purification effect of ethanol is impactful, since deprotection step may also be undertaken using this particular selection of solvent—making for an efficiency in the whole production process.

A second set of recrystallization trials (2010-R, 250 mg scale, Purity 93.5%) according to the table below was undertaken.

| | | Full Dissolution | | |
|---|---|---|---|---|
| Nr. | Solvent & Concentration | (Y/N) | Yield | Purity |
| 1 | Ethanol 10 mL/g | Yes | 0% | N/A |
| 2 | Ethanol / Acetone | No | 12% | 99.2% |
| | 1 / 1 10 mL/g | | | |
| 3 | Isopropanol 10 mL/g | No | 52% | 98.9% |
| 4 | Ethanol 5 mL/g | No (No heating) | 44% | 98.9% |
| 5 | Acetone 10 mL/g | No (No heating) | 72% | 98.7% |

This second recrystallization highlights it is possible to make a pharmaceutical product with a satisfactory quality from the raw API.

### Processing Efficiency

The results supported a hypothesis that column chromatography may not be essential to the process and it was possible to elucidate a more direct method of manufacture, which would have advantages.

To this end a set of deprotection trials involving the two available processes and various reaction treatment procedures was performed on raw Intermediate 3 having both TBDMS and Boc protecting groups present (2010-Q, 350 mg scale, SM Purity 90%).

| | Deprotection | Additional | Stirring | Stirring | | |
|---|---|---|---|---|---|---|
| Nr. | procedure | solvent | Time | Temperature | Yield | Purity |
| 1 | 4M HCl/dioxane | TBME | 30 Min | RT | 67% | 98.0% |
| 2 | 4M HCl/dioxane | Acetone | 30 Min | RT | 25% | 98.4% |
| 3 | 4M HCl/dioxane | Acetone | 30 Min | 0° C. | 42% | 98.5% |
| 4 | AcCl/EtOH | TBME | 30 Min | RT | 67% | 98.7% |
| 5 | AcCl/EtOH | TBME | 30 Min | 0° C. | 81% | 98.5% |
| 6 | AcCl/EtOH | Acetone | 30 Min | RT | 12% | 99.2% |
| 7 | AcCl/EtOH | Acetone | 30 Min | 0° C. | 25% | 98.6% |
| 8 | AcCl/EtOH | / | 30 Min | 0° C. | 67% | 98.2% |

It was confirmed that the new compound produced via this method is also much easier to isolate, as it crystallizes in the reaction mixture during deprotection (in both dioxane and ethanol pathway).

In conclusion, the applicant has determined it is possible to directly isolate a double substituted compound in a satisfactory purity directly after deprotection of raw intermediate 3 and without performing column chromatography. Although yields are quite moderate they remain relatively comparable, being in the same range or higher than the much more laborious chromatography/deprotection procedure previously used in the prior art processes to produce a different compound.

The process developed to generate the example product of the invention comprises a shorter synthesis pathway than provided for manufacturing a potentially useful Gemcitabine product to date. In particular, in the new method generated by the applicant, the last Boc-deprotection step is now possible at room temperature, which is highly advantageous; in the longer 6-step process described in the art, a far lower temperature is required to avoid Isobutyric moiety hydrolysis.

### Solubility Data

The solubility of the example mono-hydrochloride salt (LR-06-B.HCl) was determined in standard water and in buffer solution by pH 1.2, 4.5 and 6.9. In each case, a first estimation was performed by adding small amounts of solvents until dissolution of the solid (50 mg), followed by a second estimation performed through the addition of small amounts of solid to a determined quantity of solvent until saturation was reached (suspension observed after 15 min). This second estimation was undertaken twice for each solution.

| | Estimation | Estimation | Estimation | Mean |
|---|---|---|---|---|
| Solution | 1 | 2a | 2b | solubility |
| Water | 257 mg/mL | 463 mg/mL | 462 mg/mL | 394 mg/mL |
| Buffer pH 1.2 | 207 mg/mL | 217 mg/mL | 208 mg/mL | 211 mg/mL |
| Buffer pH 4.5 | 213 mg/mL | 236 mg/mL | 233 mg/mL | 227 mg/mL |
| Buffer pH 6.8 | 241 mg/mL | 302 mg/mL | 280 mg/mL | 275 mg/mL |

By comparison, solubility of the triple-substituted Gemcitabine in HCL salt form, as described in the art, typically measured only 170 mg/mL in water, which differs significantly to the present example embodiment, suggesting making the same salt selection (HCL) is not an obvious choice.

This testing indicates methods for solubilizing the compounds may be unnecessary or limited, avoiding significant additions of excipients that appeared necessary with the triple pro drug.

### Stability Data

To determine potential use in formulation development, powder and aqueous stability studies of the example compound (LR-06-B.HCL) were undertaken:

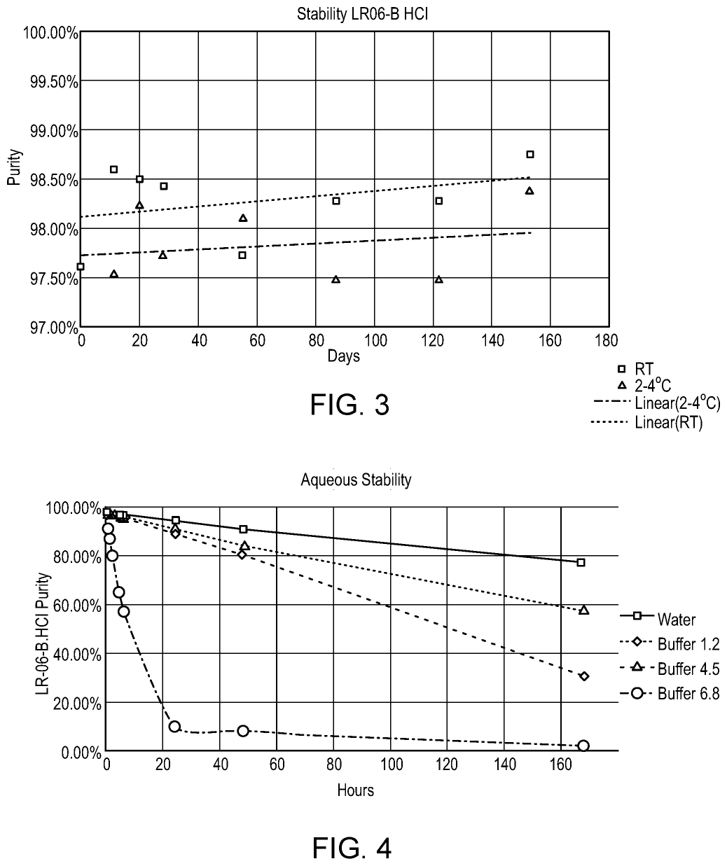

First, a powder form of the API was tested and shown in FIG. 3, after 3 months' storage at either RT or 2-4° C., no significant change was measured in either case, indicating a stable and versatile product, which is more likely to be useful in further pharmaceutical production than previously found.

Stability of LR-06-B.HCl in aqueous media at RT was also evaluated:

| Time | Water | pH 1.2 | pH 4.5 | pH 6.8 |
|---|---|---|---|---|
| 0 (API Powder) | 97.21% | 97.21% | 97.21% | 97.21% |

| | | | | | |
|---|---|---|---|---|---|
| 30 | Min | 97.06% | 97.03% | 96.45% | 91.08% |
| 1 | hour | 96.98% | 96.83% | 96.35% | 87.32% |
| 2 | hours | 96.89% | 96.42% | 96.24% | 80.70% |
| 4 | hours | 96.62% | 95.71% | 95.46% | 65.65% |
| 6 | hours | 96.42% | 95.14% | 94.87% | 57.40% |
| 1 | day | 94.45% | 88.40% | 90.73% | 9.48% |
| 2 | days | 91.07% | 80.05% | 84.24% | 7.48% |
| 7 | days | 77.12% | 30.60% | 57.17% | 1.77% |

A significant short-term degradation of the API in aqueous media was observed, see FIG. 4.

Regarding to the MS profile, most of the observed impurities concern a valproic moiety cleavage and/or valine moiety migration/cleavage. The UV-profile suggests that the core of the molecule is not impacted by the degradation. In agreement with earlier observation, a lower acidity medium will likely result in a higher ratio of valine hydrolysis.

The physical and chemical properties of the two salts (present example and triple substituted prior art example) are surprisingly different. Based on the poor stability of the same selected salt, it is apparent that the salt choice is not impactful but rather the selection 2 as compared to three functional groups on the API.

### Additional Example Compounds

Additional examples of a substituted Gemcitabine HCL (where R₂ remains H) were produced and identified during these processing investigations. The example compounds are defined by the combination of two groups selected at R₁ and R₃, respectively:
Valproic acid (2-propylpentanoic acid) (R₁) and Valproic acid (R₃), the compound of that embodiment being: (2R,3R,5R)-4,4-difluoro-2 (hydroxymethyl)-5-(2-oxo 4-(2propylpentanamido)-1,2-dihydropyrimidin-1-yl)oxolan-3-yl 2-propylpentanoate; or
Valproic acid and H, the compound of that embodiment being N-(1-((2R,4R,5R)-3,3-difluoro-4-hydroxy-5-(hydroxymethyl)oxolan-2-yl)-2-oxo-1,2-dihydropyrimidin-4-yl)-2-propylpentanamide; or
Boc Valine (2-(tert-butoxycarbonyl)-amino-3-methylbutanoic acid) and H, the compound of that embodiment being: tert-butyl (1-((1-((2R,4R,5R)-3,3-difluoro-4-hydroxy-5-(hydroxymethyl)oxolan-2-yl)-2-oxo-1,2-dihydropyrimidin-4-yl)amino)-3-methyl-1-oxobutan-2-yl)carbamate; or
Valine (2-amino-3-methylbutanoic acid) and H, the compound of that embodiment being 2-amino-N-(1-((2R,4R,5R)-3,3-difluoro-4-hydroxy-5-(hydroxymethyl)oxolan-2-yl)-2-oxo-1,2-dihydropyrimidin-4-yl)-3-methylbutanamide hydrochloride; or
BocValine and BocValine, the compound of that embodiment being (2R,3R,5R)-5-(4-(2-((tert-butoxycarbonyl)amino)-3-methylbutanamido)-2-oxo-1,2-dihydropyrimidin-1-yl)-4,4-difluoro-2-(hydroxymethyl)oxolan-3-yl (2S)-2-(tert-butoxycarbonyl) amino-3-methylbutanoate;
Valine and Valine, the compound of that embodiment being (2R,3R,5R)-5-(4-(2-amino-3-methylbutanamido)-2-oxo-1,2-dihydropyrimidin-1-yl)-4,4-difluoro-2-(hydroxymethyl)oxolan-3-yl (2S)-2-amino-3-methylbutanoate dihydrochloride; or
BocValine and Isobutyric acid (2-methylpropanoic acid), the compound of that embodiment being (2R,3R,5R)-5-(4-(2-((tert-butoxycarbonyl)amino)-3-methylbutanamido)-2-oxo-1,2-dihydropyrimidin-1-yl)-4,4-difluoro-2-(hydroxymethyl)oxolan-3-yl isobutyrate; or
Valine and Isobutyric acid, the compound of that embodiment being (2R,3R,5R)-5-(4-(2-amino-3-methylbutanamido)-2-oxo-1,2-dihydropyrimidin-1-yl)-4,4-difluoro-2-(hydroxymethyl)oxolan-3-yl isobutyrate hydrochloride; or
Valproic acid and Isobutyric acid, the compound of that embodiment being (2R,3R,5R)-4,4-difluoro-2-(hydroxymethyl)-5-(2-oxo-4-(2-propylpentanamido)-1,2-dihydropyrimidin-1-yl)oxolan-3-yl isobutyrate.

LC-MS and NMR were performed to confirm production and a 100 mg sample was stored.

### Bioavailability

Three most promising LR06 formulation in a new dog PK study to assess oral bioavailability against i.v. administered Gemcitabine (LR06b is administered orally, Gemcitabine is measured systemically in the blood, the percentage bioavailability of Gemcitabine is determined by comparing with i.v. administered Gemcitabine—data pending).

The formulation of LR06b for oral administration in dogs and observed that the oral bioavailability was at least as good as the referenced case (triple substituted) (over 50% measured as i.v. Gemcitabine comparison).

1. A compound of the Formula (I) or an N-oxide thereof, or a pharmaceutically acceptable salt, solvate, polymorph, tautomer, stereoisomer, an isotopic form, or a prodrug of said compound of Formula (I) or N-oxide thereof: 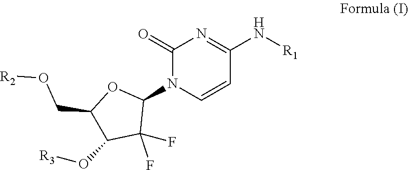
wherein, R₁ is selected from 2-propylpentanoic acid, 2-(tert-butoxycarbonyl)-amino-3-methylbutanoic acid, and 2-amino-3-methylbutanoic acid;
R₂ is H; and
R₃ is selected from 2-propylpentanoic acid, 2-(tert-butoxycarbonyl)-amino-3-methylbutanoic acid, and 2-methylpropanoic acid.

2. A compound selected from:
(2R,3R,5R)-4,4-difluoro-2 (hydroxymethyl)-5-(2-oxo 4-(2propylpentanamido)-1,2-dihydropyrimidin-1-yl)oxolan-3-yl 2-propylpentanoate;
N-(1-((2R,4R,5R)-3,3-difluoro-4-hydroxy-5-(hydroxymethyl)oxolan-2-yl)-2-oxo-1,2-dihydropyrimidin-4-yl)-2-propylpentanamide;
tert-butyl (1-((1-((2R,4R,5R)-3,3-difluoro-4-hydroxy-5-(hydroxymethyl)oxolan-2-yl)-2-oxo-1,2-dihydropyrimidin-4-yl)amino)-3-methyl-1-oxobutan-2-yl)carbamate;
2-amino-N-(1-((2R,4R,5R)-3,3-difluoro-4-hydroxy-5-(hydroxymethyl)oxolan-2-yl)-2-oxo-1,2-dihydropyrimidin-4-yl)-3-methylbutanamide hydrochloride;
(2R,3R,5R)-5-(4-(2-((tert-butoxycarbonyl)amino)-3-methylbutanamido)-2-oxo-1,2-dihydropyrimidin-1-yl)-4,4-difluoro-2-(hydroxymethyl)oxolan-3-yl (2S)-2-(tert-butoxycarbonyl) amino-3-methylbutanoate;
(2R,3R,5R)-5-(4-(2-amino-3-methylbutanamido)-2-oxo-1,2-dihydropyrimidin-1-yl)-4,4-difluoro-2-(hydroxymethyl)oxolan-3-yl (2S)-2-amino-3-methylbutanoate dihydrochloride;
(2R,3R,5R)-5-(4-(2-((tert-butoxycarbonyl)amino)-3-methylbutanamido)-2-oxo-1,2-dihydropyrimidin-1-yl)-4,4-difluoro-2-(hydroxymethyl)oxolan-3-yl isobutyrate;
(2R,3R,5R)-5-(4-(2-amino-3-methylbutanamido)-2-oxo-1,2-dihydropyrimidin-1-yl)-4,4-difluoro-2-(hydroxymethyl)oxolan-3-yl isobutyrate hydrochloride; and
(2R,3R,5R)-4,4-difluoro-2-(hydroxymethyl)-5-(2-oxo-4-(2-propylpentanamido)-1,2-dihydropyrimidin-1-yl)oxolan-3-yl isobutyrate.

3. The compound of any previous claim wherein the salt is a hydrochloride salt.

4. A compound represented by Formula (II): 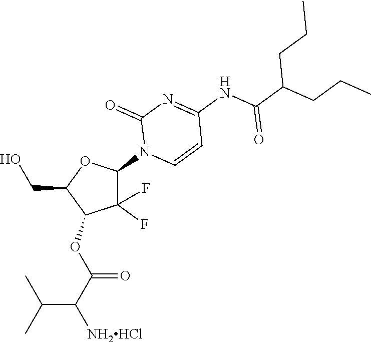
or namely: (2R,3R,5R)-4,4-difluoro-2-(hydroxymethyl)-5-(2-oxo-4-(2-propylpentanamido)-1,2-dihydropyrimidin-1-yl)oxolan-3-yl (2S)-2-amino-3-methylbutanoate hydrochloride.

5. A pharmaceutical composition, comprising a formulation including one or more pharmaceutically acceptable excipients and the compound according to any of claims 1 to 4, optionally wherein the formulation is a powder.

6. The pharmaceutical composition according to claim 5 for use as a medicament.

7. The pharmaceutical composition according to claim 6 for use in the treatment of an autoimmune condition or a neoplastic disease, optionally a pim-overexpressed neoplastic disease including but not limited to, leukemia, lymphoma, multiple myeloma, prostate cancer, pancreatic cancer, gastric cancer, colon cancer, or liver cancer.

8. Use of the compound according to claims 1 to 4, in the manufacture of a medicament for treating an autoimmune condition or a neoplastic disease including a pim-overexpressed neoplastic disease including but not limited to, leukemia, lymphoma, multiple myeloma, prostate cancer, pancreatic cancer, gastric cancer, colon cancer, or liver cancer.

9. A method of treating an autoimmune disease, a neoplastic disease and optionally a Pim-overexpressed neoplastic disease including but not limited to leukemia, lymphoma, multiple myeloma, prostate cancer, pancreatic cancer, gastric cancer, colon cancer, or liver cancer, by administering to a subject in need thereof an effective amount of the compound of claims 1 to 4, or the composition of claim 5.

10. A method of producing a compound, as provided in any of claims 1 to 4 and in accordance with formula (I) below: 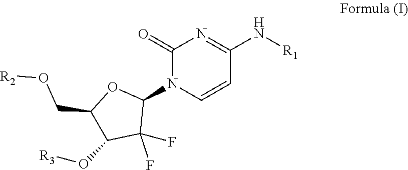
comprising the steps:
selectively protecting a primary alcohol group at R₂, to produce a first intermediate (1);
coupling a peptide on the amino group of the first intermediate at R₁, to produce a second intermediate (2);
undertaking a peptide-like esterification of the second intermediate (2) at R₃, to produce a third intermediate (3); and
deprotecting the third intermediate (3).

11. The method of producing a compound in accordance with claim 10, wherein the protecting step is performed with silylated reactants (TBDMSCl).

12. The method of producing a compound in accordance with claim 10 or 11, wherein the coupling step is performed using EDCl.HCl/HOBt, optionally wherein the coupling step is undertaken in the absence of an additional base, preferably in the absence of DIPEA or TEA.

13. The method of producing a compound in accordance with any claim from 10 to 12, wherein the esterification step is performed using EDCl.HCl/HOBt as coupling agent and/or DMAP as catalyst.

14. The method of producing a compound in accordance with any claim from 10 to 13, wherein the deprotecting step is undertaken with 4M HCl/dioxane or AcCl/EtOH.

15. The method of producing a compound in accordance with any claim from 10 to 14, further comprising an isolation step including a precipitation step using and anti-solvent, optionally TBME.
